(19) United States
(12) Patent Application Publication
(10) Pub. No.: US 2021/0387126 A1
(43) Pub. Date: Dec. 16, 2021

(54) POROUS MATERIALS FOR TREATING CONTAMINANTS

(71) Applicant: Metalmark Innovations, Inc., Cambridge, MA (US)

(72) Inventors: Elijah SHIRMAN, Winchester, MA (US); Tanya Shirman, Winchester, MA (US); Sissi Liu, Arlington, MA (US)

(21) Appl. No.: 17/348,464

(22) Filed: Jun. 15, 2021

Related U.S. Application Data
(60) Provisional application No. 63/039,376, filed on Jun. 15, 2020.

(51) Int. Cl. B01D 46/00; C02F 1/72; A61L 9/00; B01D 46/24; C02F 1/00; B01D 39/16; B01D 39/20

(52) U.S. Cl. CPC B01D 46/0061; C02F 1/725; A61L 9/00; B01D 46/24491; B01D 46/2476; C02F 1/001; B82Y 35/00; B01D 39/2027; B01D 39/2055; A61L 2209/21; C02F 2305/10; C02F 2305/08; B01D 39/1676

(57) ABSTRACT

In one aspect, a material structure is disclosed, which includes a macroscopic porous substrate configured to receive a flow of a medium for passage of at least a portion thereof through the porous substrate. At least one porous coating is disposed on at least a portion of an inner surface of the porous substrate, wherein the porous coating comprises a matrix having a plurality of interconnected passages. The porous substrate and the coating are configured to treat at least one contaminant, if any, present in the flowing medium.

### CROSS-REFERENCE TO RELATED APPLICATIONS

The present application claims priority to U.S. Provisional Application No. 63/039,376 filed on Jun. 15, 2020, which application is incorporated herein by reference in its entirety.

### BACKGROUND

The present disclosure relates to materials with structural microscopic and macroscopic features and related compositions that are designed to efficiently treat (e.g., inactivate) viruses and other pathogenic microorganisms, as well as treat gaseous and particulate contaminants.

Air decontamination from particulates and airborne pathogens including viruses, represents a significant technological challenge using common technologies such as filtration, sorption, ionization, or photodegradation due to their limited ability to remove or inactivate contaminants or pathogens without forming secondary toxins.

Aerosol transmission routes play a significant role in the spread of various pathogens (Nature https://www.nature.com/articles/d41586-020-00974-w, 2020; and Yan et al. Proc. Nat Acad. Sci. USA, 2018, 115, 1082). Bioaerosols can include bacteria, viruses, fungi, algae, and dust mites. In addition, biological materials such as pollen, endotoxins, proteins, and animal excreta form aerosols. Airborne pathogens are almost always embedded in droplets along with various levels and types of organic and inorganic materials. This heterogeneity represents a significant challenge and needs to be taken into account in the development and evaluation of air decontamination technologies.

Numerous technologies for bioaerosol control have been in development and usage as measures to mitigate the airborne transmission of infectious diseases (Pluschke P., 2018; Zhang Y., 2011; and Luengas A., 2015). One of the common methods is based on physical removal of bioaerosols from an air stream (e.g. filter, electrostatic precipitator, etc.).

Existing technologies for inactivation of airborne bioaerosols include photocatalysis (mostly using a UV light), photoelectrocatalysis, microwave irradiation, and cold plasma. These approaches have various efficiencies, yet their safety remains questionable since they release hazardous by-products, e.g. radicals and ozone due to the use of UV-light and ionizing energy.

Viral inactivation through thermal heating has been considered a safe and reliable method; however, it is not well suited for gas phase aerosolized matter as the temperature requirements are significantly higher than for more commonly studied liquid or solid phases. Aerosolized viruses and other pathogens (present in droplets as compared to individual microorganisms) demonstrate an increased thermal and chemical stability in comparison to those suspended in liquid or attached to solid surfaces due to low characteristic exposure times in an air purification system, which are typically well below 1 sec (typically below 0.1 sec).

Numerous studies have shown that large fractions of viruses can survive temperatures exceeding 250° C. at sub-second residence times (Grinshpun et al., 2010). In summary, heat-treatment, as well as UV-based solutions, require much longer residence times than those required for typical air handling systems.

Development of novel non-toxic materials for air decontamination, which can be incorporated into personal protective equipment and air purification devices for building, aircrafts, and vehicles, to provide safer environments, is needed. There is also a need for such materials that can be employed to treat and/or decontaminate a liquid medium.

Summary

Disclosed is material structures that possess unique properties applicable, for example, to antiviral air purification. In some embodiments, the antiviral function is combined with the removal of a wide range of other hard-to-address aerosols and particulate matters such as ultrafine particles (i.e., particulates below 0.1 microns, PM0.1) and PM1 and volatile pollutants.

The platform technology is based on scalable wet chemistry and self-assembly of nano/micro-structured building blocks.

In some embodiments the material disclosed herein is used in the form of coatings on macroscopic porous substrates.

As discussed in detail below, in certain embodiments, the combination of the microstructure and composition of the coating with the structure and composition of the macroscopic substrate provide a synergistic effect for the inactivation of contaminants, such as viruses, and other pathogenic microorganisms, and/or removal of gaseous and particulate contaminants.

In one aspect, a material structure is disclosed, which includes a macroscopic porous substrate having one or more channels, said porous substrate having at least one inlet port for receiving a flow of a medium and at least one outlet port through which at least a portion of the received medium can exit the porous substrate. At least one porous coating is disposed on at least a portion of an inner surface of the one or more channels, wherein the porous coating comprises a matrix having a plurality of interconnected passages (e.g., pores), and a plurality of active sites disposed on an internal surface of at least one of said interconnected passages (e.g., pores).

In some embodiments, the macroscopic porous substrate and the porous coating are configured to cooperatively treat at least one contaminant in the flowing medium. In some embodiments, the macroscopic porous structure, said coating and optionally said active sites are configured to treat at least a portion of one or more bioaerosols, if any, in said medium flowing through said channels of the macroscopic porous substrate.

In some embodiments, the macroscopic structure, the coating and optionally the active sites are configured to treat at least a portion of one or more types of particulates, e.g., in the range of 10-300 nm, if any, present in the flowing medium. In some embodiments, the macroscopic porous substrate, the coating and optionally the active sites are configured to treat at least a portion of one or more types of pathogenic organisms, if any, present in the flowing medium.

In some embodiments, at least one pathogenic microorganism includes any of one or more viruses, bacteria, and fungi present in the flowing medium.

In some embodiments, the medium includes contaminated air. In some such embodiments, the contaminant includes an airborne contaminant. In some embodiments, the medium includes a liquid. In some embodiments, the liquid includes any of a water-based liquid, an aqueous dispersion, an organic liquid, an organic dispersion, and an ionic liquid.

In some embodiments, the macroscopic porous substrate, the coating and optionally the active sites are configured to treat at least one contaminant present in the liquid. In some embodiments, said at least one contaminant includes any of a virus, a bacterium, and fungi flowing through the channels of the macroscopic porous substrate.

In some embodiments, the at least one contaminant can be in any of a solid phase, gas phase, and liquid phase. In various configurations, the present teachings can be applied for filtration or separation of liquid and/or gas streams. By way of example, streams having different solid, gas and/or liquid solutes can be separated. By way of another example, streams having different-sized solid particles can be separated.

In some embodiments, the macroscopic porous substrate, the porous coating and optionally the active sites are configured to provide entrapment of one or more contaminants, if present, in the flowing medium. In some embodiments, the contaminants can comprise any of gases, particulates, aerosols, bioaerosols, pathogens, volatile organic compounds (e.g., vapor phase or condensed phase), or the like, or any combinations thereof. In some embodiments, the macroscopic porous substrate together with the porous coating of a structure according to the present teachings can prolong the transit time of contaminants (e.g., pathogens such as viruses) through the structure, thus facilitating treatment (e.g., deactivation) of such contaminants. Herein, prolonging the transit time of contaminants can refer to increasing the transit time relative to a bulk residence time of the flowing medium, which can be represented by the volume of the macroscopic porous substrate divided by the volumetric flow rate of the flowing medium.

In some embodiments, the pores of the coating exhibit a geometry, a surface roughness and/or a size configured to facilitate the entrapment or retardation of said at least one contaminant (e.g., particulate). For example, the RMS roughness (root-mean-squared roughness) can be in the range of 1 nm to about 20 nm, e.g., in a range of about 5 nm to about 10 nm.

In some embodiments, the coating can exhibit any of inverse opal structure, a sponge-like, or a gyroid geometry. In some embodiments, the coating can exhibit a thickness in a range of about 1 to about 200 micrometers, e.g., in a range of about 10 micrometers to about 150 micrometers, or in a range of about 50 micrometers to about 100 micrometers.

In some embodiments, the interconnected pores of the coating exhibit a cross-sectional dimension in a range of about 10 nm to about 20 microns, or in a range of about 50 nm to about 20 microns, e.g., in a range of about 100 nm to about 10 microns, or in a range of about 200 nm to about 10 microns, or in a range of about 250 nm to about 5 microns, or in a range of about 50 nm to about 300 nm, or in a range of about 300 nm to about 5 microns, or in a range of about 1 micron to about 2 microns.

In some embodiments, the interconnected pores of the coating exhibit a surface area in the range of about 10 m²/g to about 1000 m²/g, e.g., in a range of about 100 m²/g to about 500 m²/g.

In some embodiments, the interconnected pores of the coating exhibit a cross-sectional size that is equal to or greater than an average size of said at least one contaminant, e.g., a particulate or a pathogen (e.g., a virus), and less than about a hundred times of the average size of said at least one contaminant.

In some embodiments, the pores can have a cross-sectional size in a range of about one to about 200 times, e.g., in a range of about 1 to about 100 times, or in a range of about 1.5 to about 100 times, or in a range of about 2 to about 100 times, an average size of at least one target contaminant (e.g., particulate).

In some embodiments, the coating includes any of oxides, mixed oxides, mixed oxides of elements from one or more groups I, II, III, IV V, VI, zeolites, oxohydroxides, aluminates, silicates, alumosilicates, titanates, oxometallates, metal-organic frameworks, vanadia, silica, alumina, titania, zirconia, hafnia, nickel oxide, cobalt oxide, cobalt oxide, tin oxide, manganese oxide, magnesium oxide, noble metal oxides, platinum group metal oxides, molybdenum oxides, tungsten oxides, rhenium oxides, tantalum oxide, niobium oxide, chromium oxides, scandium, yttrium, lanthanum, thorium, rare earth oxides or a combination thereof.

In some embodiments, the coating includes any of a synthetic polymer, a natural polymer, a bio-polymer, or a combination thereof.

In some embodiments, the channels of the macroscopic substrate are sized and shaped to cause turbulence in flow of said medium, e.g. air, flowing through said channels. In some embodiments, the macroscopic channels and the coating are configured such that the flow of the medium through the channels results in treatment of at least 70% of said at least one contaminant. In some embodiments, the macroscopic channels and the coating are configured such that the flow of the medium through the channels results in treatment of at least about 80%, or at least 90%, or at least about 95%, or at least about 99%, or at least 100%, of said at least one contaminant.

In some embodiments the structure of macroscopic substrate is configured to force air to flow through its porous internal walls (e.g. diesel particulate filter) by, for example, capping the end wall of at least some of flow channels such that the air is forced to flow from the channel that is capped into an adjacent channel through a porous walls separating those channels.

In some embodiments, the coating includes a continuous film. In other embodiments, the coating includes a plurality of discontinuous surface segments.

In some embodiments, the coating can completely fill pores of macroscopic substrate. For example, in some embodiments, at least a portion of a substrate's pores or channels can be completely filled with porous coating.

In some embodiments, at least a portion of an inner surface of said porous coating is catalytically active so as to treat said at least one contaminant. For example, in some embodiments, instead of or in addition to incorporating active sites on one or more portions of the coating, the coating itself, or at least a surface portion thereof, can be formed of a catalytically active material or antiviral or antibacterial material. Some examples of such catalytically active materials include, without limitation, manganese oxide, copper oxide, and silver oxide.

In some embodiments, one or more channels of the porous substrate have an average cross-sectional dimension in a range of about 50 microns to about 10,000 microns, e.g., in a range of about 100 microns to about 5,000 microns, or in a range of about 200 microns to about 1,000 microns. In some embodiments, one or more channels exhibit a length in a range of about 1 mm to about 1 m, e.g., in a range of about 100 mm to about 50 cm, or in a range of about 200 mm to about 100 cm.

In some embodiments, the porous macroscopic substrate includes any of a ceramic, a metal, a metallic alloy, a carbide, a metal felt, FeCrAl, natural clay, a polymeric material and combinations thereof. By way of example, the ceramic can include a cordierite.

In some embodiments, the porous macroscopic substrate can be in the form of a monolith, metal mesh structure, ceramic or metal foam structure, polymeric foam, packed-bed structure, or HEPA type filter. In some embodiments, commercially available substrates or filters can be used as the porous macroscopic substrate, onto which the microscopic coatings are applied.

In some embodiments, the channels of the porous macroscopic substrate exhibit a geometry selected from the group consisting of a cylinder, a mesh, a foam, a spiral profile, a bead, and woven or non-woven fibers-like structure. The woven structure is weaved by interlacing warp and weft threads, when the nonwoven structure is made by bonding fibers together by physical means.

In some embodiments, the channels of the porous macroscopic substrate are arranged relative to one another as any of a plurality of parallel channels, randomly oriented channels, interconnected or isolated channels, a sponge-like configuration, a corrugated geometry, a spiral geometry and any combination thereof.

In some embodiments, the active sites comprise any of a metal, one or more metal alloys, a multimetallic species, a metal cation, a metal sulfide, a binary metal salt, a metal salt of transition metals, a complex metal salt, a metal salt of an organic acid, a metal salt of inorganic acid, a metal salt of a complex acid, a base, an acid, organometallic complexes, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, metal oxides, mixed metal oxides, iron oxides, cobalt oxides, nickel oxides, manganese oxide, magnesium oxide, noble metal oxides, ruthenium oxides, rhodium oxides, palladium oxides, osmium oxides, iridium oxides, platinum oxides, copper oxides, silver oxides gold oxides, vanadium oxides, niobium oxide, tantalum oxide, chromium oxide, molybdenum oxide, tungsten oxide, scandium oxide, yttrium oxide, lanthanum oxide, rare earth metal oxide, and combinations thereof.

In some embodiments, the active sites include a plurality of nanoparticles. By way of example, the nanoparticles can include any of metal, multiple metals, a metal alloy, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, bimetals, metal alloys, a metal oxide, a mixed metal oxide, a metal sulfide, a binary metal salt, a complex metal salt, a metal salt of an organic acid, a metal salt of inorganic acid, a metal salt of a complex acid, a base, an acid, an organometallic compound, a coordination compound, one or more platinum group metal oxides, silica, alumina, iron oxides, cobalt oxides, nickel oxides, ruthenium oxides, rhodium oxides, palladium oxides, osmium oxides, iridium oxides, platinum oxides, copper oxides, silver oxides, gold oxides, vanadium oxides, zirconium oxide, cerium oxide, manganese oxide, magnesium oxide, niobium oxide, tantalum oxide, chromium oxide, molybdenum oxide, tungsten oxide, scandium oxide, yttrium oxide, lanthanum oxide, rare earth metal oxide, and combinations thereof.

In some embodiments, the active sites include a biological agent. By way of example, the biological agent can be a protein that is chemically or physically coupled to an internal surface portion of said coating. In some embodiments, the protein can be an enzyme.

In some embodiments, the active sites include semiconductor nanoparticles that are doped with any of group III and group V elements, or a combination thereof.

In some embodiments, the active sites can be activated via any of heat and/or radiation. By way of example, the active sites can be activated by raising a temperature thereof to a range of about 15° C. to about 350° C. In some cases, the active sites and/or the rest of the material structure are maintained in this temperature range as the structure is employed for purifying a medium flowing through the structure. By way of example, the radiation for activating the active sites can have a wavelength in a range of microwave, UV, visible and IR portions of the electromagnetic spectrum and combinations thereof. For example, the activating radiation can have a wavelength in a range of about 160 nm to about 1,500 nm.

In some embodiments, the active sites are configured to provide at least one of a catalytic, a photonic, an antimicrobial, a light-absorbing, a light-emitting, a stimuli responsiveness, an adsorption, a desorption property and combination thereof. In some embodiments, the catalytic property includes any of a photocatalytic and an electrocatalytic property.

In a related aspect, a material structure is disclosed, which includes a macroscopic porous substrate, where the porous substrate can receive a flow of a medium for passage of at least a portion thereof through the porous substrate. For example, the porous substrate can include at least one inlet port for receiving a flow of a medium and at least one outlet port through which at least a portion of the received medium can exit the porous substrate. In some embodiments, the porous substrate can be in the form of a matrix of a plurality of interconnected passages (herein also referred to as channels) through which a received flow of a medium can propagate. In some embodiments, at least one porous coating is disposed on at least a portion of at least an inner surface of the porous substrate. For example, such a coating can be disposed on at least a portion of an inner surface of at least one of a plurality of interconnected passages of a porous substrate. In some embodiments, the porous coating comprises a matrix having a plurality of interconnected passages (e.g., pores). The macroscopic porous substrate and the coating are configured to treat at least one contaminant (e.g., particulate), if any, present said flowing medium.

In some embodiments, at least a portion of an inner surface of at least one of said passages (e.g., pores) of the coating comprises a catalytically active material suitable for treating said contaminant . By way of example, the catalytically active material can be any of a thermally, a photocatalytically and an electrocatalytically active material.

In some embodiments, one or more active sites are disposed on an inner surface of at least one of the passages (e.g., pores) of the coating.

In some embodiments, the macroscopic porous structure, the coating and optionally the active sites are configured to treat at least a portion of one or more bioaerosols, if any, present in said flowing medium.

In some embodiments, the macroscopic structure, the coating and optionally the active sites are configured to treat at least a portion of one or more types of particulates, if any, present in the flowing medium. By way of example, in some embodiments, the size range of the particulates that can be treated is about 1 nm to about 1 micron. In some embodiments, the size range of the particulars to be treated can be in a range of about 5 nm to about 500 nm, or in a range of about 10 nm to about 300 nm. As discussed further below, it has been discovered that by tuning a number of parameters of the structure (e.g., the pore sizes of the macroscopic substrate and/or the porous coating), a structure in accordance with some embodiments can be configured to filter (e.g., inactivate, remove) particulates with a size in a range of about 10 nm to about 300 nm.

In some embodiments, the macroscopic porous substrate, the coating and optionally the active sites are configured to treat at least a portion of one or more types of pathogenic organisms, if any, present in the flowing medium.

By way of example, the pathogenic microorganism comprises any of one or more viruses, bacteria, and fungi present in the flowing medium. By way of example, the medium includes contaminated air. In some embodiments, the contaminant includes at least one airborne contaminant. The contaminant can be a pathogen, such as a virus, a bacterium and fungi.

By way of example, the medium can be a liquid, such as a water-based liquid, an aqueous dispersion, an organic liquid, an organic dispersion, and an ionic liquid.

In some embodiments, the macroscopic porous substrate and the porous coating are configured to provide entrapment (as discussed below, the term “entrapment” is used herein to indicate a permanent or a temporary capture of a contaminant) and/or retardation of said at least one contaminant. In some such embodiments, the pores of the coating can exhibit a geometry, a surface roughness and a size configured to facilitate said entrapment and/or retardation of said at least one particulate. For example, the porous coating can exhibit any of an inverse opal geometry, a sponge-like geometry, or a gyroid geometry. For example, the RMS roughness (root-mean-squared roughness) can be in the range of 1 nm to about 20 nm.

In some embodiments, the coating can exhibit a thickness in a range of about 1 to about 200 micrometers, e.g., in a range of about 10 micrometers to about 100 micrometers. In some embodiments, the interconnected pores of the coating can exhibit a cross-sectional dimension in a range of about 100 nm to about 20 microns. In some embodiments, the interconnected pores of the coating can exhibit a cross-sectional dimension in a range of about 200 nm to about 10 microns. In some embodiments, the cross-sectional dimension of the interconnected pores of the coating can be in a range of about 300 nm to about 5 microns. In some such embodiments, the interconnected pores of the coating can exhibit a surface area in a range of about 10 m²/g to about 500 m²/g.

Further, in some such embodiments, the interconnected pores of the coating exhibit a cross-sectional size that is equal to or greater than an average size of said at least one contaminant and less than about a hundred times of the average size of said at least one contaminant.

By way of example, the coating can include any of oxides, mixed oxides, mixed oxides of elements from one or more groups I, II, III, IV V, VI, zeolites, oxohydroxides, aluminates, silicates, alumosilicates, titanates, oxometallates, metal-organic frameworks, vanadia, silica, alumina, titania, zirconia, hafnia, nickel oxide, cobalt oxide, cobalt oxide, tin oxide, manganese oxide, magnesium oxide, noble metal oxides, platinum group metal oxides, molybdenum oxides, tungsten oxides, rhenium oxides, tantalum oxide, niobium oxide, chromium oxides, scandium, yttrium, lanthanum, thorium, rare earth oxides or a combination thereof.

In some embodiments, the coating can be any of a synthetic polymer, a natural polymer, a bio-polymer or a combination thereof. In some embodiments, the channels of the macroscopic substrate are sized and shaped to cause turbulence in flow of the medium flowing through said channels. By way of example, the dimensions of the channels can be selected to render the Reynolds number based on the representative cross-sectional diameter of the channels to be greater than about 2,000. In some embodiments, the Reynolds number can be greater than 2,900, or 3,000, or 10,000, or 1×10⁵, or 5×10⁵. In some embodiments, flow obstructions, corners, forward-facing or backward-facing steps, and/or surface roughness can be included in the channels to facilitate transitioning to the turbulent flows.

In some embodiments, the macroscopic channels and the coating are configured such that the flow of the medium through the channels results in treatment of at least 70%, or at least 80%, or at least 90%, or at least 95%, or at least 99%, or 100%, of said at least one contaminant.

In some embodiments, the coating includes a continuous film. In other embodiments, the coating can be in the form of a plurality of discontinuous segments.

In some embodiments, the one or more channels of the porous substrate have an average cross-sectional dimension in a range of about 50 microns to about 10,000 microns.

In some embodiments, the one or more channels of the macroscopic porous substrate can exhibit a length in a range of about 1 mm to about 1 m. The channels can have a variety of geometrical shapes and can be arranged relative to one another according to a variety of different patterns. For example, at least one of the channels can have a straight or an arcuate shape. Further, in some embodiments, one or more of the channels can be parallel to one another, or can be intersecting so as to provide a pattern of interconnected channels.

In some embodiments, the porous macroscopic substrate can include any of a ceramic, a metal, a metallic alloy, a carbide, a metal felt, a metal foam, FeCrAl, natural clay, a polymeric material and combinations thereof. In some such embodiments, the ceramic includes a cordierite.

In some such embodiments, the channels of the porous macroscopic substrate exhibit a geometry selected from the group consisting of a cylinder, a mesh, a foam, a spiral profile, a bead, and woven or non-woven fibers-like structure. The woven structure can be weaved by interlacing warp and weft threads, when the nonwoven structure is made by bonding fibers together by physical means.

The channels of the porous macroscopic substrate are arranged relative to one another as any of a plurality of parallel channels, randomly oriented channels, interconnected or isolated channels, according to a sponge-like configuration, according to a corrugated geometry, according to a spiral geometry and any combination thereof.

In some embodiments, the active sites include any of a metal, one or more metal alloys, a multimetallic species, a metal cation, a metal sulfide, a binary metal salt, a metal salt of transition metals, a complex metal salt, a metal salt of an organic acid, a metal salt of inorganic acid, a metal salt of a complex acid, a base, an acid, organometallic complexes, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, metal oxides, mixed metal oxides, iron oxides, cobalt oxides, nickel oxides, manganese oxide, magnesium oxide, noble metal oxides, ruthenium oxides, rhodium oxides, palladium oxides, osmium oxides, iridium oxides, platinum oxides, copper oxides, silver oxides gold oxides, vanadium oxides, niobium oxide, tantalum oxide, chromium oxide, molybdenum oxide, tungsten oxide, scandium oxide, yttrium oxide, lanthanum oxide, rare earth metal oxide, and combinations thereof.

In some embodiments, the active sites include a plurality of nanoparticles. By way of example, the nanoparticles can include any of metal, multiple metals, a metal alloy, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, bimetals, metal alloys, a metal oxide, a mixed metal oxide, a metal sulfide, a binary metal salt, a complex metal salt, a metal salt of an organic acid, a metal salt of inorganic acid, a metal salt of a complex acid, a base, an acid, an organometallic compound, a coordination compound, one or more platinum group metal oxides, silica, alumina, iron oxides, cobalt oxides, nickel oxides, ruthenium oxides, rhodium oxides, palladium oxides, osmium oxides, iridium oxides, platinum oxides, copper oxides, silver oxides, gold oxides, vanadium oxides, zirconium oxide, cerium oxide, manganese oxide, magnesium oxide, niobium oxide, tantalum oxide, chromium oxide, molybdenum oxide, tungsten oxide, scandium oxide, yttrium oxide, lanthanum oxide, rare earth metal oxide, and combinations thereof.

In some embodiments, the active sites can include a biological agent. The biological agent can include, for example, a protein that is chemically or physically coupled to an internal surface portion of the coating. By way of example, the protein can be an enzyme.

In some embodiments, the active sites can include semiconductor nanoparticles that are doped with any of group III and group V elements, or a combination thereof. In some embodiments, the active sites can be activated via heat and/or radiation. By way of example, the active sites can be activated by raising a temperature thereof to a range of about 15° C. to about 350° C. In some embodiments, the active sites can be activated by exposing them to radiation having a wavelength in a range of any of microwave, ultraviolet (UV), visible and infrared (IR) portions of the electromagnetic spectrum and combinations thereof. By way of example, the activating radiation can have a wavelength in a range of about 160 nm to about 1500 nm.

In some embodiments, the coating is configured to provide at least one of a catalytic, a photonic, an antimicrobial, a light-absorbing, a light-emitting, a stimuli responsiveness, an adsorption, a desorption property and combination thereof. By way of example, the catalytic property can include any of a photocatalytic and an electrocatalytic property.

In a related aspect, a method of fabricating a material structure for treatment of at least one target contaminant in a flowing medium is disclosed, which includes forming a composite coating component (slurry) by mixing a templating component, an active component and a coating precursor, applying the composite coating component (slurry) onto a at least a portion of an inner wall of at least one channel of a macroscopic porous substrate comprising a plurality of interconnected channels, at least partially removing the templating component from said composite coating such that the coating precursor forms a matrix comprising an interconnected network of pores having a cross-sectional size in a range of about one to about 100 times an average size of the target contaminant, wherein said active component forms a plurality of active sites on at least a portion of an inner surface of said interconnected pores.

In some embodiments, the step of forming the composite coating component includes dipping the macroscopic substrate into a dispersion of the composite coating components and said slurry including , the templating component, the active component and the coating precursor (porous matrix precursor).

In some embodiments, the active component is coupled to the templating component via any of a covalent interaction, coordination complexation, an ionic bonding, and a Van-der-Waals interaction.

In some embodiments, the composite coating component is formulated to produce dispersed composite micro-particles (See, Route B in FIG. 9). In some embodiments, the composite coating component includes a homogeneous dispersion.

In some embodiments, the active component includes a catalytic nanoparticle.

In a related aspect, a method of fabricating a material structure is disclosed, which includes forming a composite coating component by mixing a templating component and a coating precursor, applying the composite coating component onto at least a portion of an inner wall of at least one channel of a macroscopic porous substrate comprising a plurality of interconnected channels, at least partially removing the templating component from the composite coating such that the coating precursor forms a matrix comprising an interconnected network of pores, and modifying the porous coating with an active component so as to generate a plurality of active sites on at least a portion of an inner surface of said interconnected pores (See, Route A in FIG. 9).

In some embodiments, the pores can have a cross-sectional size in a range of about one to about 200 times, e.g., in a range of about 1 to about 100 times, or in a range of about 1.5 to about 100 times, or in a range of about 2 to about 100 times, an average size of at least one target particulate (e.g., contaminant).

In some embodiments, the step of modifying the coating with an active component includes employing any of chemical modification, vapor deposition, spray coating and atomic layer deposition.

In some embodiments of the above method, the active sites can include any of a metal, one or more metal alloys, a multimetallic species, metal cations, a metal sulfide, a binary metal salt, a metal salt of transition metals, a complex metal salt, a metal salt of an organic acid, a metal salt of inorganic acid, a metal salt of a complex acid, a base, an acid, organometallic complexes, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, metal oxides, mixed metal oxides, iron oxides, cobalt oxides, nickel oxides, manganese oxide, magnesium oxide, noble metal oxides, ruthenium oxides, rhodium oxides, palladium oxides, osmium oxides, iridium oxides, platinum oxides, copper oxides, silver oxides gold oxides, vanadium oxides, niobium oxide, tantalum oxide, chromium oxide, molybdenum oxide, tungsten oxide, scandium oxide, yttrium oxide, lanthanum oxide, rare earth metal oxide, and combinations thereof. In some embodiments, the active sites can include a biological agent, such as those described above.

In some embodiments of the above method, the active sites can include semiconductor particles that are doped with any of group III and group V elements, or a combination thereof.

In some embodiments of the above method, the active sites can be activated via any of heat and radiation, e.g., in a manner discussed above.

In some embodiments of the above method, the active sites are configured to provide at least one of a catalytic, a photocatalytic, an electrocatalytic, a photonic, an antimicrobial, a light-absorbing, a light-emitting, a stimuli responsiveness, an adsorption, a desorption property and combination thereof.

In a related aspect, a method of fabricating a material structure for treatment of at least one target contaminant in a flowing medium is disclosed, which includes forming a composite coating component by mixing a templating component, an active component and a coating precursor, formulating the composite coating component into a plurality of dispersed composite micro-particles, using the dispersed composite micro-particles to form a composite microparticles slurry, applying the composite micro-particles slurry onto at least a portion of an inner wall of at least one channel of a macroscopic porous substrate comprising a plurality of interconnected channels to form a composite coating, and at least partially removing the templating component from said composite coating such that the coating precursor forms a matrix comprising an interconnected network of pores, wherein said active component forms a plurality of active sites on at least a portion of an inner surface of said interconnected pores.

In some embodiments of the above method, the step of applying the composite microparticles slurry onto at least a portion of an inner wall of said macroscopic substrate comprises dipping said macroscopic substrate into the composite microparticles slurry to form a composite coating.

In some embodiments of the above method, the step of applying the composite microparticles slurry onto at least a portion of an inner wall of the macroscopic substrate comprises spray-coating at least a portion of the inner wall of said macroscopic substrate with a said composite microparticles slurry to form a composite coating.

In some embodiments of the above method, the step of applying the composite microparticles slurry onto at least a portion of an inner wall of the macroscopic porous substrate comprises electrodeposition of said composite coating components to form a composite coating on at least a said portion of an inner wall of said macroscopic substrate.

In some embodiments, the templating component comprises any of a polymer, a hydrogel, an organogel, random and block copolymers, branched, star and dendritic polymers, supramolecular polymers and combinations thereof. In some embodiments, the templating component comprises polymer colloids, such as polystyrene, polyurethane, poly(methyl methacrylate), polyacrylate, poly(alkyl acrylate), substituted polyalkylacrylate, polystyrene, poly(divinylbenzene), polyvinylpyrrolidone, poly(vinylalcohol), polyacrylamide, poly(ethylene oxide), polyvinyl chloride, polyvinylidene fluoride, polytetrafluoroethylene, other halogenated polymers, and combinations thereof.

In some embodiments, the templating component includes any of a polymeric fibers, biopolymer fibers, fibers with organometallic composition, supramolecular self-assembled fibers, and a combination thereof. In some embodiments, the templating component comprises any of natural materials, such as cellulose, natural rubber (e.g. latex), wool, cotton, silk, linen, hemp, flax, and feather fiber, any of natural or synthetic fabrics and textiles, and combinations thereof. In some embodiments, the process of removing said templating component include calcination, dissolution, etching, evaporation, sublimation, phase-separation, and combinations thereof.

In some embodiments, a coating precursor comprises any of oxides, mixed oxides, mixed metal oxides, silica, alumina, titania, vanadia, zirconia, nickel oxide, cobalt oxide, tin oxide, manganese oxide, magnesium oxide, chromium oxides, noble metal oxides, platinum group metal oxides, molybdenum oxides, zeolites, oxyhydroxides, bihemite, aluminates, silicates, inorganic sol-gels, metals, metal alloys, organometallic compounds, synthetic or natural polymers, rare earth oxides or a combination thereof.

In some embodiments, the step of formulating the composite coating component into a plurality of dispersed composite microparticles comprises spray drying. In some embodiments, the step of formulating the composite coating component into plurality of dispersed composite microparticles comprises mixing with surfactant, detergents, wetting agents, emulsifiers, foaming agents, and dispersants. In some embodiments, the step of forming a composite microparticles slurry comprises an addition of binder.

In some embodiments, the binder comprises one of metal oxides, mixed metal oxide, silica, alumina, titania, metal oxide colloids, inorganic colloids, inorganic particles, oxohydroxides, aluminates, silicates, alumosilicates, titanates, oxometallates, vanadia, zirconia, hafnia, nickel oxide, cobalt oxide, tin oxide, manganese oxide, magnesium oxide, noble metal oxides, platinum group metal oxides, molybdenum oxides, tungsten oxides, rhenium oxides, tantalum oxide, niobium oxide, chromium oxides, scandium, yttrium, lanthanum, thorium, rare earth oxides or a combination thereof.

In a related aspect, a method of fabricating a material structure for treatment of at least one target contaminant in a flowing medium is disclosed, which includes forming a composite coating component by mixing a templating component and a coating precursor, formulating the composite coating component into plurality of dispersed composite micro-particles, using said dispersed composite micro-particles to form a composite microparticles slurry, applying the composite micro-particles slurry onto at least a portion of an inner wall of at least one channel of a macroscopic porous substrate comprising a plurality of interconnected channels to form a composite coating, at least partially removing the templating component from said composite coating such that the coating precursor forms a matrix comprising an interconnected network of pores to produce porous coating, modifying said porous coating with an active component so as to generate a plurality of active sites on at least a portion of an inner surface of said interconnected pores. The various methods of applying a slurry as well as various materials disclosed herein can be employed in various embodiments of this method.

Further understanding of various aspects of the present disclosure can be obtained by reference to the following detailed description in conjunctions with the associated drawings, which are described briefly below.

### BRIEF DESCRIPTION OF THE DRAWINGS

FIG. 1A provides examples of typical indoor air pollutants, their size range; and relevant target range of the present teachings (data compiled from: Pluschke P., 2018; Zhang Y., 2011; and Luengas A., 2015);

FIG. 1B depicts typical removal efficiencies of fibrous media HVAC filters rated by the MERV metric (EPA, 2018);

FIG. 2A depicts a typical air purification unit;

FIG. 2B is a schematic representation of bioaerosol inactivation and particulates treatment mechanism according to an embodiment of the present teachings;

FIC. 2C is a magnified schematic view of a fragment of a porous interconnected coating according to an embodiment with particulates propagating through it and interacting with surface active sites;

FIG. 3 depicts example of air purification unit according to an embodiment, including various examples of macroscopic structures with characteristic cross sectional diameter of the channel, and a magnified view of a nanostructured coating with characteristic pore size;

FIG. 4 depicts example of a material structure in which the macroscopic substrate (MS) includes a plurality of discrete elements such as beads coated with porous coating;

FIG. 5 depicts different morphologies of the coating;

FIG. 6 shows examples of coatings on ceramic substrates;

FIG. 7 shows examples of coating on metallic substrates;

FIG. 8 shows the results of two test trials, and an average and a standard deviation thereof for inactivating live viruses entrained in air flowing through the catalytic unit consisting of ceramic substrate coated with catalytically active nanoporous coating; and

FIG. 9 is an example of a fabrication method of the structured material according to an embodiment.

### DETAILED DESCRIPTION

The present teachings disclose a material structure for treatment of bio-contaminants such as viruses, and at least partial removal of particulate and gaseous contaminants from a medium, such as air and water. The treatment of polluted streams refers to processes aimed to remove, filter or inactivate the contaminants.

As discussed above, various technologies for controlling particulates, bioaerosol and other contaminants have been in development and usage as measures to mitigate the levels of particulate matter and the airborne transmission of infectious diseases (Pluschke P., 2018; Zhang Y., 2011; and Luengas A., 2015). One of the common methods is based on physical removal of these contaminants from an air stream (e.g. filter, electrostatic precipitator, etc.). However, as shown in FIG. IA, conventional filtration techniques work poorly for particulate matters (PM) under about 1μm in size. Filtration technologies, including HEPA-based filters, are not well suited for the task due to their limited ability to capture viruses and absence of an active mechanism to inactivate them, resulting in the possible release of virulent particles (potentially at elevated concentrations) due to the fluctuations in pressure, temperature, or humidity, as well as degradation of the filter material (Kemp et al., 2001; and Zhang et al., 2011). Viral particles, which can be described as “molecular machines,” are amongst the smallest particles (approximately 20 to 200 nm) that can be transported in a bioaerosol. For example, Rhinovirus is about 30 nm, and coronaviruses can be as small as approximately 70 to 150 nm (Wikipedia).

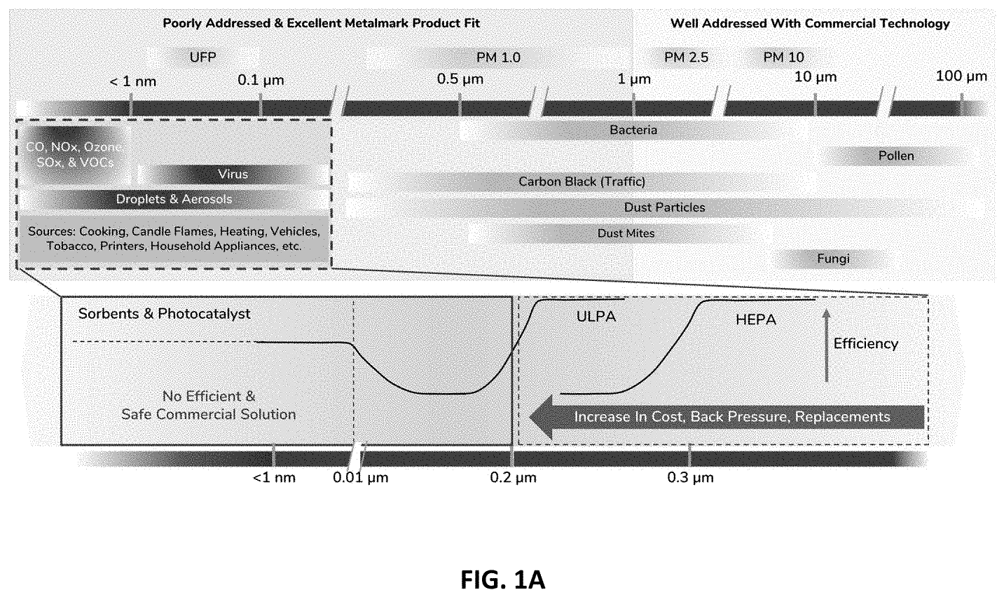

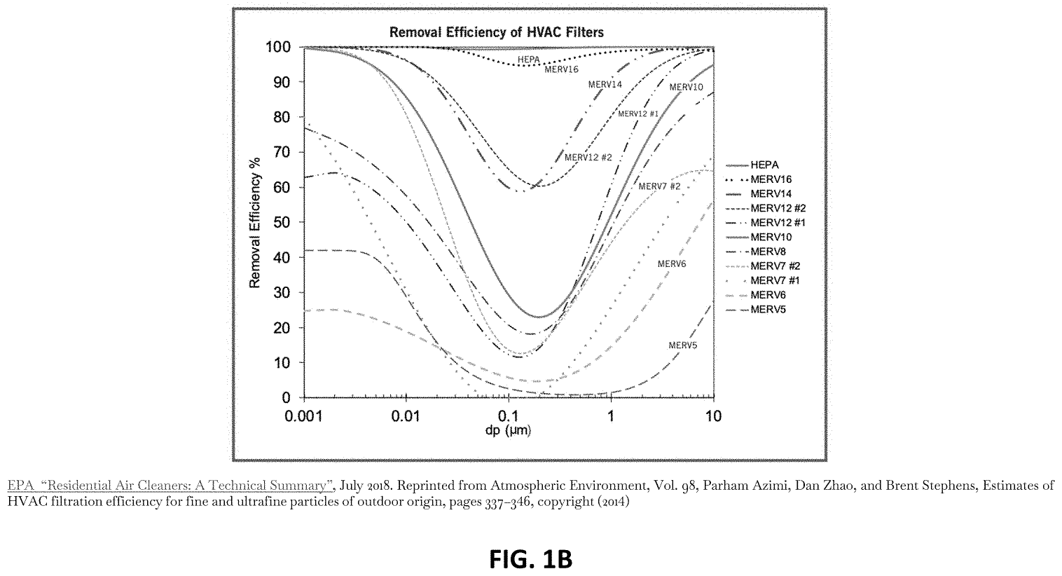

As shown in FIG. 1B, recent studies found that the most penetrating particle size for filters is between 0.1-0.3 μm, precisely the range of the majority of viruses and other fine particulates (EPA's Technical Report on Residential Air Cleaners, July 2018). Even if the virus is captured on a filter, it can remain active for an extended period and thus a filter itself can become a source for additional contamination or a medium for bacteria and fungi growth. The survivability of infectious microorganisms on filters is a well-known phenomenon (Kempa, P. C. et al. “Survival and growth of microorganisms on air filtration media during initial loading” Atmospheric Environment, 2001, 35, 4739-4749; Bortolassi, A. C. C. et al. “Characterization and evaluate the efficiency of different filter media in removing nanoparticles” Separation and Purification Technology, 2016; and Guo, J. et al.“Bacterial community analysis of floor dust and HEPA filters in air purifiers used in office rooms in ILAS, Beijing” Scientific Reports, 2020, 10, 6417).

In view of these needs for treating bioaerosols and particulates with a size range of, e.g., about 10 nm to about 500 nm more effectively, the present teachings provide novel non-toxic materials for gas and/or liquid decontamination, which can be incorporated into personal protective equipment and air and/or water purification systems for building, aircrafts, and vehicles, to provide safer environments.

As discussed below, a material structure according to the present teachings can include a plurality of porous components with different ranges of pore sizes. For example, in many embodiments, a material structure according to the present teachings can include a porous substrate having a plurality of channels through which a medium (e.g., air or water) can flow and a porous coating that is disposed on at least a portion of an internal surface of at least one of the channels of the substrate. In many embodiments, an average size of the pores of the porous coating is smaller than an average size of the channels of the substrate. For example, in embodiments in which the channels of the porous substrate and the pores of the porous coating have a substantially circular cross-sectional shape, the average diameter of the pores of the porous coating can be less than an average diameter of the channels of the porous substrate. In this manner, a material structure can include multiple porous components exhibiting pores in different size ranges. For example, in some embodiments, the pores of the porous coating can have an average size in the nanometer regime while the channels of the porous substrate can have a size in the micrometer to sub-millimeter regime.

The term “particulate,” as used herein, refers to a variety of inorganic and organic material structures, including naturally-occurring and artificial material structures, such a variety of microorganisms (e.g., bacteria and/or viruses). By way of example, such a particulate can have a size of at most about 10 microns or below (e.g., “PM10”), or at most about 2.5 microns or below (e.g., “PM2.5”), or at most about 1 micron or below (e.g., “PM1”), or at most about 300 nm or below. The term “ultrafine particulate,” typically refers to a particulate having a size of at most about 0.1 microns (“PM0.1”) or below.

The terms “treat” and “treatment” are used herein to refer to oxidation, reduction, inactivation, degradation, and filtration (e.g., removal) (or a combination thereof) of a contaminant (e.g., gas, vapor, particulate matter, aerosol, bioaerosol, or pathogen) from a medium (e.g., a gas or liquid medium), including a flowing medium, e.g., in the form of a polluted stream.

The term “entrapment,” as used herein, refers to a permanent or temporary capture of a contaminant (e.g., a particulate) by a structure according to the present teachings.

The term “retardation,” as used herein, refers to an increase in the transit time of a contaminant as it passes through a structure according to the present teachings relative to the transit time as it passes through a putative straight channel having substantially the same length of the structure at the same flow rate therethrough.

The terms “pore,” “passage,” “passageway,” and “channel” are herein used interchangeably to refer to a material structure having at least one opening for receiving the flow.

The pores can be of a spherical or non-spherical shape, e.g., linear, curvilinear, tortuous, bifurcating, or branched cavity that can provide an enclosure or a surface that is exposed to the flow.

The term “back pressure” is used herein to refer to a pressure drop or loss in a flow of a medium across the material structure.

The term “size” as used herein refers to a cross-sectional dimension, e.g., a dimension, such as a maximum dimension, perpendicular to an elongated dimension (e.g., length)) of a pore or a channel (such as a diameter of a pore or a channel), e.g., in the case of a high aspect ratio pore (when the ratio between the long and the short dimension of a pore is greater than 1.5). As such, in the embodiments discussed below, a pore or a channel can be characterized by one or more of its cross-sectional dimensions and its length.

In many embodiments, the compositions and materials disclosed herein offer a multi-pronged mechanism for treatment of pathogens and other particles such as ultrafine particulate matter (PM) or PM0.1-1. As described below, in some embodiments, this goal can be achieved via rational design of a structure with nano and micro scale features that includes a porous coating deposited on at least a portion of internal channels of a porous macroscopic substrate.

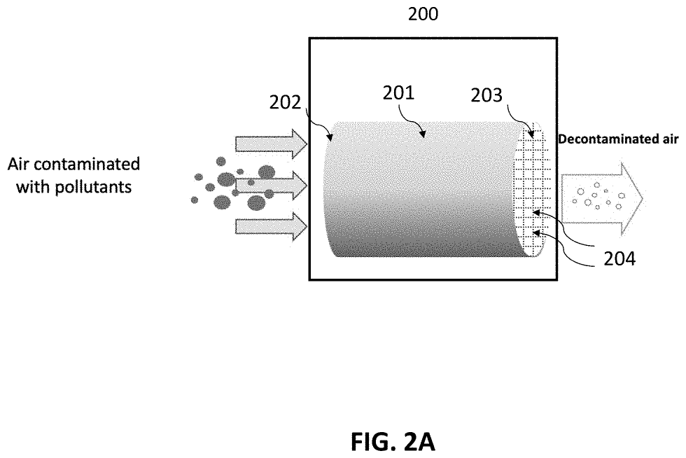

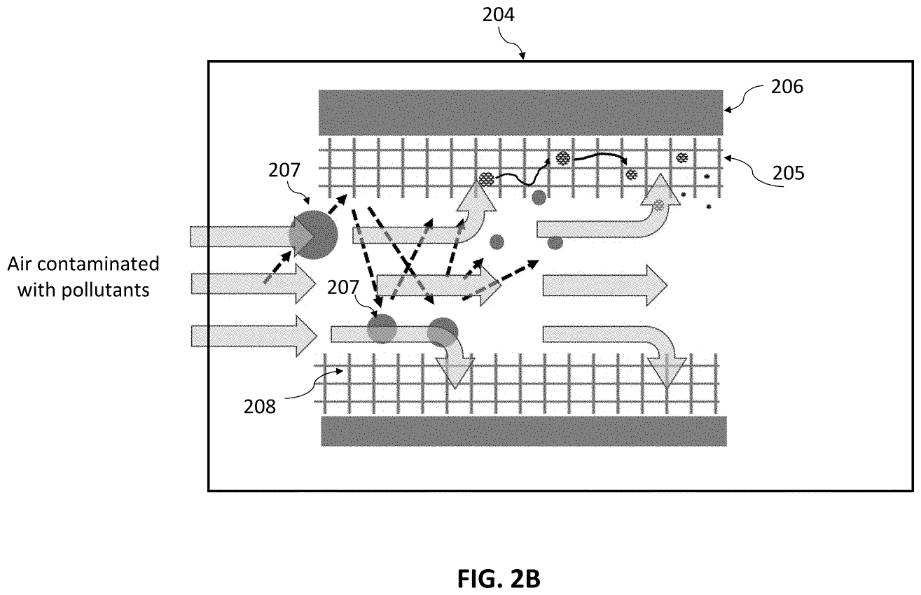

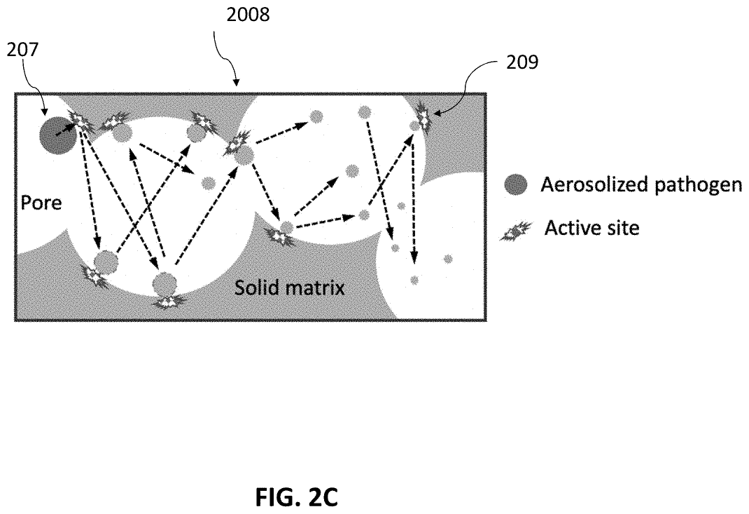

FIGS. 2A and 2B schematically depict an example of a material structure 200 and its function according to an embodiment of the present teachings. The material structure 200 includes a porous substrate 201 (herein also referred to as “a macroscopic porous substrate” and “a macroscopic porous structure” (MS)) with an inlet 202 for receiving a medium (e.g., an incoming air flow in this embodiment) in which one or more contaminants may be entrained and an outlet/exit port 203 through which at least a portion of the received medium can exit the structure. In this embodiment, the outlet/exit port 203 allows for the exit of outgoing purified air.

The macroscopic porous structure includes multiple channels 204 that are coated with a micro and/or nano-structured porous material, which includes an interconnected porous network in the form of a plurality of interconnected channels. By way of example, FIG. 2B depicts an example of one such channel 204 and its structure. A porous coating 205 is deposited on at least a portion of an inner wall 206 of the channel 204.

In use, as an air stream in which one or more contaminant particles 207 may be present flows through the channels of the macroscopic porous structure, such as the channel 204, contaminant particles 207 can hit the walls of the channel and enter the interconnected porous network of the coating 208, with pore sizes configured to trap the particles.

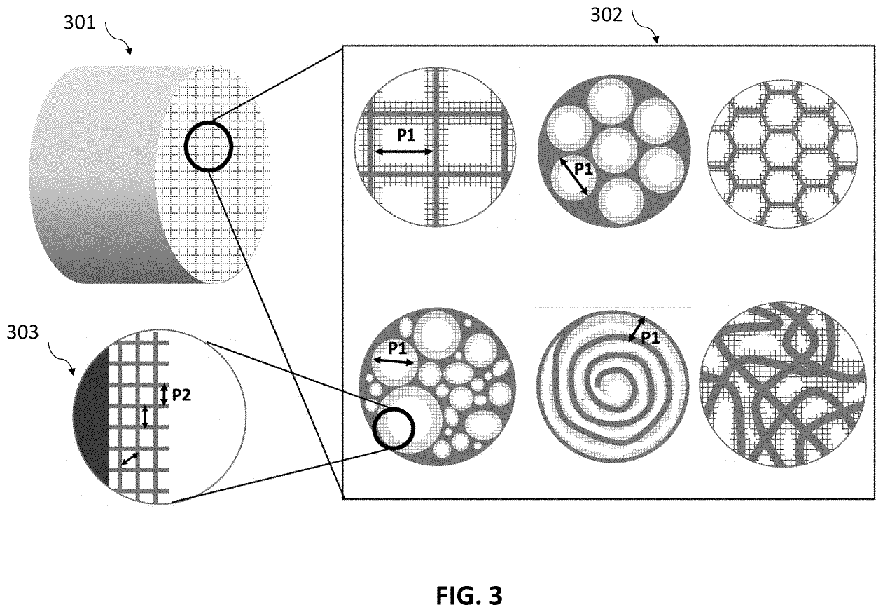

FIG. 2C shows examples of such interconnected pore design 2008 of a porous coating according to an embodiment. In this embodiment, the macroscopic porous substrate is configured to support high rates of air flow with low back pressures, which render the disclosed system particularly useful for incorporation in air handling units. For example, the MS can include a plurality of separate channels and/or a sequence of interconnected channels having characteristic cross sectional diameter, e.g., in a range of about 50 microns to about 10,000 microns, for example in a range of about 50 microns to about 1,000 microns, or in a range of about 100 microns to about 5,000 microns, or in a range of about 200 microns to about 1,000 microns. (See, e.g., P1 in FIG. 3) and an effective length (corresponding to the average path length of the airflow through the MS). The effective length can range from one (straight channels, e.g., a cordierite monolith) to 100 (one hundred) (100) of an external length of the MS (e.g. in foam form).

In some embodiments in which the macroscopic porous substrate exhibits these properties, the back pressure for a desired range of the flow rate of a medium through the structure can remain within the acceptable range.

Effective length of a channel needs to be long enough to enable sufficient interaction with the coating such that at least one contaminant entrained in a medium (such as air) flowing through the material structure can be treated, e.g., inactivated and/or removed.

In this embodiment, the coating 205 includes a plurality of active sites 209, which are described in more detail below. While in this embodiment the active sites 209 are in the form of a plurality of distinct elements, in other embodiments an entire internal surface of one or more pores of the porous coating can be in the form of an active layer that can treat one or more contaminants (e.g., pathogens, etc.). In other embodiments, the coating 205 itself can be active and treat one or more contaminants (e.g., pathogens, etc.).

In some embodiments the treatment of pollutants can be achieved through heating of the material structure. For example, in use, the material structure can be maintained at an elevated temperature, e.g., in a range of about 15° C. to about 350° C.

The described structural features and the composition of the coating in combination with the structural features of the macroscopic channels of the substrate enables enhancing one or more of the following properties of the material structure:

(1) slowing down the propagation of aerosols, viruses, other particulates, and gases through the material structure, thereby increasing the transit time thereof within the material structure,

(2) increasing the number of interactions of the propagating aerosol and or particulates with the active surface of the coating,

(3) breaking up aerosols,

(4) trapping the viruses in the intricate network of pores, and

(5) inactivating the pathogens by (a) causing oxidative damage to their structures through interaction with active sites 209 in the materials and/or (b) prolonged thermal contact with the material inside the porous coating.

In some embodiments, a material structure according to the present teachings, such as the above material structure 200, can inactivate and/or decompose a variety of airborne bio-contaminants or aerosol particles containing biological contaminants (or fragments thereof) including but not limited to viruses, bacteria, fungi, fungi spores, spore fragments, primary and secondary animal metabolites (microbial volatile organic compounds), and pollen that can originate from animal dander (cats, dogs, rabbits, rodents, birds), excretes; and moulds.

In some embodiments the passage of ultrafine particles (<0.1 μm), PM1.0 and PM2.5 of organic, inorganic or mixed origin through a structure according to the present teachings can result in the decomposition of such particles.

Herein, when the particulates are non-spherical, the size thereof is not limited to a specific definition of the particle size, and may be described by various definitions used in the field to describe the representative dimensions of the particulate matter. By way of example, an aerodynamic diameter of an irregular particle may refer to a diameter of hypothetical spherical particle with a density of 1,000 kg/m³ having the same settling velocity as the irregular particle. A mobility diameter may refer to an equivalent diameter that corresponds to the same electrical mobility of a singly charged spherical particle. A volume equivalent diameter may refer to a diameter of a sphere having an equivalent volume. Further, when the particle sizes have a polydisperse distribution, the particle diameter may be represented by a log-normal mean diameter of the size distribution, which can be referred to as “a mean diameter” or “an average size.”

In some embodiments, a material structure 200 comprises a coating 205 disposed on a macroscopic structure 301 (MS) with various geometries of their channels 302 as shown in FIG.

3. The channels can be arranged in a parallel manner, or can have a sponge or foam-like structure with a wide distribution of channel lengths and cross-sectional diameters. For example, in some embodiments, the channel can have an effective length equal to the external length of the MS and the channels can have cross-sectional diameters in a range of about 50 microns to about 10,000 microns, e.g., about 1,000 microns to about 5,000 microns.

In some embodiments the open channels have parallel geometry such as those in monolithic cordierite honeycombs.

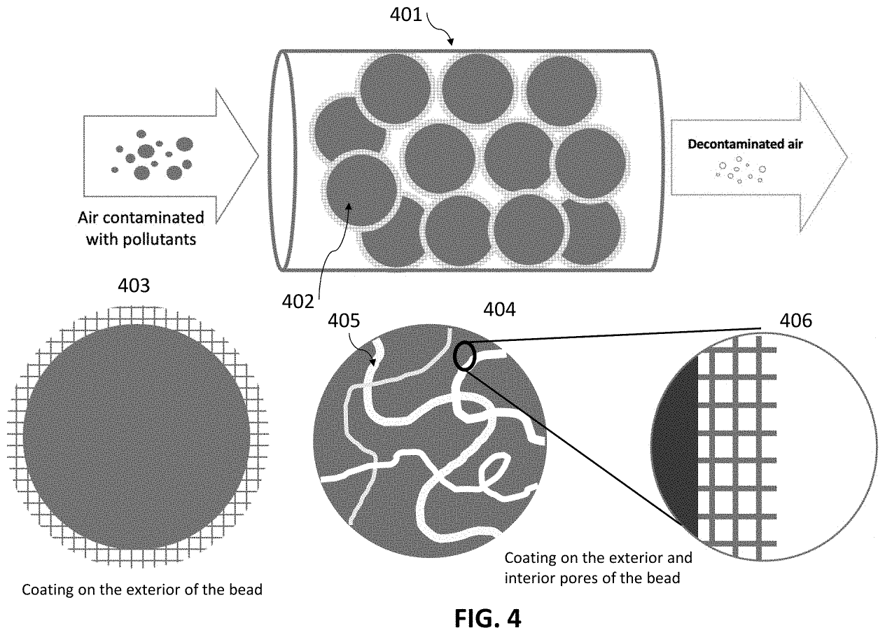

In other embodiments, an MS 401 can include discrete elements such as beads 402 having an average cross-sectional size ranging from 5 microns to about 5 mm (FIG. 4). In some embodiments, the beads 402 can be coated with a microscopically porous coating, as described previously, to form composite beads 403.

Yet in other embodiments, the beads 402 can have a porous structure characterized by a plurality of channels 405. By way of example, the channels can have an effective length corresponding to the average path length of the airflow through the MS and a cross-sectional dimension in a range of about 300 nm to about 1,000 microns.

In these embodiments, one or more of the beads can be externally coated with a microscopically porous coating, such as the coatings discussed above. Such a coating can cover at least a portion of an exterior surface 403, at least a portion of an interior pore 404-406, or both the exterior and the interior pore of the bead.

In some embodiments, the beads can include, for example, metal oxides, mixed metal oxides, metals, alumina, silica, titania, zeolites, alumina silicates and combination thereof.

In some embodiments, the length of a channel of the MS can be at least 100 times greater than its typical diameter. For example, in such embodiments, channels with a diameter of 0.5 mm have a length of at least 50 mm. This, in combination with the structural features of the coating (e.g. porosity and roughness) can further enhance the delayed propagation (i.e., increase the residence time) of contaminants, such as ultrafine particulates or pathogens, through the material structure, thus facilitating their ultimate entrapment and/or deactivation.

In some embodiments, a material structure according to the present teachings can facilitate the formation of local turbulent flows in the vicinity of the surface coating and the diffusion of contaminants, such as viruses, bioaerosols, and particulates, into the porous coating (perpendicular to the axis of the air stream), thus greatly enhancing the time of interaction and the effective physical and chemical contact with the porous coating, e.g., one or more catalytic active sites provided on the inner channels of the coating.

A material structure according to the present teachings can also be effectively employed for treating contaminants present in a liquid medium, e.g., water. For example, the channels of the porous substrate together with the pores of the coating, as discussed above, can cooperatively treat one or more contaminants present in a liquid flow.

As noted above, in some embodiments, a material structure according to the present teachings can include one or more active sites that are disposed on the inner surface of one or more pores of the porous coating. In other embodiments, a material structure according to the present teachings does not include such active sites.

Nonetheless, such material structures without active sites can still treat a variety of contaminants, such as pathogens, including viruses and microbes. For example, in case of pathogen particles, the temporal entrapment of pathogens (or other contaminants) inside the pores of such a coating without active sites, can increase the transit time of the particles. Such an increase in the transit time of the particles can be sufficient for inactivation of the pathogen, e.g., due to thermal and/or radiation effects when the material structure is maintained at an elevated temperature, e.g., in a range of about 60° C. to about 500° C. The rational design of coating morphology and structure, pore geometry and size according to the present teachings, e.g., such as those discussed above, can make this type of inactivation mechanism efficient. Even without active sites or forming a coating of catalytically active material, the structure according to such embodiments can still treat contaminants due to the thermal and/or radiation effect.

A structure according to the present teachings can be used in a variety of different applications. Some examples of such applications include, without limitation, personal protective equipment; air purification systems/devices for buildings, aircrafts, and vehicles; water purification systems/devices for municipal facilities, buildings, off-shore facilities, and watercrafts; dialysis systems; and general membranes, sensors, and smart coatings for any filtration, purification, separation, or size exclusion applications.

### Coatings

In some embodiments, the sizes (e.g., cross-sectional diameters) of the pores of the coating applied to the MS (the microscopic interconnected pores, P2 in FIG. 3) are larger than but less than a hundred times larger than the typical size of at least one target contaminant of interest. For example, if the typical size of a virus is 100 nm, then the pore sizes of the coating (e.g., an average pore size) can be in a range of about 100 nm and 50 microns. In some embodiments, to ensure that the target contaminants are received within the pores, the pore sizes of the coating can be in a range of about 100 nm and 20 microns, or in a range of about 200 nm and 20 microns. Herein, the pore sizes of the coating may be defined by any representative size of the pores. For example, it may be defined by an average pore size, a minimum (e.g., a bottom 10^{th}-percentile), or a median pore size.

In some embodiments, the pores can have a cross-sectional size in a range of about one to about 200 times, e.g., in a range of about 1 to about 100 times, or in a range of about 1.5 to about 100 times, or in a range of about 2 to about 100 times, an average size of at least one target contaminant (e.g., particulate).

In other embodiments, if the typical size of a virus is 100 nm then the pore sizes of the coating (e.g., an average pore size) can be in a range of about 100 nm and 10 microns. In yet other embodiments, if the typical size of a virus is 100 nm then the pore sizes of the coating (e.g., an average pore size) can be in a range of about 300 nm and 5 microns.

The pore structure and size can be rationally designed and tuned to a wide variety of pollutants sizes.

In the context of this disclosure, the average pore size refers to e.g. the pore diameter or a cross-sectional dimension (e.g., the largest or the average cross-sectional dimension), e.g., in the case of a high aspect ratio pore (when the ratio between the long and the short dimension of a pore is greater than 1.5).

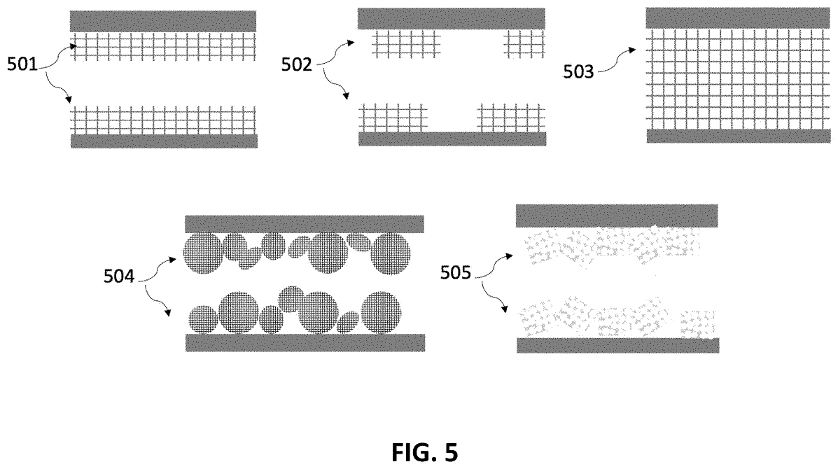

The coating can have different morphologies as shown in FIG. 5.

In some embodiments, the coating can comprise a continuous film 501. In other embodiments, the coating comprises a discontinuous film 502 or can completely fill the pore of the macroscopic structure 503. In other embodiments, the coating comprises a collection of discrete macroscopic particulates with the porous structure as described above, including a plurality of materials islands, clusters or aggregates 504 and 505.

According to the present teachings, the coating can be made from a variety of materials or mixtures of materials. By way of example, in some embodiments, the materials can comprise one or more metal oxides, metals (such as gold, palladium, platinum, silver, copper, rhodium, ruthenium, rhenium, titanium, osmium, iridium, iron, cobalt, or nickel, or a combination thereof), semiconductors (such as silicon, germanium, tin, silicon doped with group III or V elements, germanium doped with group III or V elements, tin doped with group III or V elements, or a combination thereof), a metal sulfide, a metal chalcogenide, a metal nitride, a metal pnictide and combination thereof.

In other embodiments, the coating can comprise one of silica, alumina, titania, zirconia, ceria, hafnia, vanadia, beryllia, noble metal oxides, platinum group metal oxides, titania, tin oxide, molybdenum oxide, tungsten oxide, rhenium oxide, tantalum oxide, niobium oxide, chromium oxide, scandium oxide, yttria, lanthanum oxide, thorium oxide, uranium oxide, other rare earth oxides, and a combination thereof.

In certain embodiments, the coating can include one or more organometallic complexes (such as metal organic frameworks), inorganic polymers (such as silicone), organometallic complexes, or combinations thereof, covalent, non-covalent and supramolecular polymers (such as polystyrene, polyurethane, hydrogels, and organogels), natural materials, a protein- or polysaccharide-based material, silk fibroin, chitin, shellac, cellulose, chitosan, alginate, gelatin, or a mixture thereof, and mixtures thereof.

The coating can be designed, for example, to be catalytically active, stimuli-responsive, chemically robust, degradable, and/or exhibit specific optical, thermal, mechanical, sorption, release, and/or acoustic properties. By way of example, such coatings can include catalytically active metal oxides such as titania, copper oxide, ceria, zirconia, manganese oxide, and nickel oxide. In certain embodiments, the coating can interact with light in a way that it becomes active toward pollutant treatment (e.g., photocatalysis, photothermal catalysis, or photoelectrocatalysis). In some embodiments the composition of the coating can be modified to provide enhanced mechanical properties and robustness by utilizing mechanically robust materials such as alumina, tungsten oxide, and metal alloys. Yet in other embodiments the specific optical properties can be introduced through design of porosity and pore ordering in the coating (e.g. photonic structures such as inverse opals).

In some embodiments, the coating can include one or more materials that facilitate/enhance the adsorption of bioaerosols, particulates, gaseous contaminants, and other pollutants. In some embodiments, such enhanced adsorption properties can be due to the presence of chemical functional groups on the surface of coating (e.g. amine or thiol), and the coating composition (e.g. metal oxides, silica, zeolites, activated carbon).

In some embodiments, the coating can exhibit sorption (both adsorption and absorption) properties including sorption of gases (e.g. VOCs, CO₂, CO, ammonia and its derivatives), particulate matter and microorganisms (e.g. bacteria, viruses, etc.).

In some embodiments, the coating exhibits both sorption and catalytic activity. For example, the coating can include one or more metal oxides with surface properties designed with increased affinity toward certain pollutants (hydroxylated surface or surface with amine functions to improve the adsorption of polar molecule such as formaldehyde or alcohol or hydrophilic particle) and elemental composition with catalytic activity toward treatment of pollutants (e.g. nickel oxide, palladium oxide, mixed metal oxides).

In some embodiments, the functionality of the active sites can originate, at least partially, from the morphological features of the coating surface such as roughness. For example, the rough surface can comprise spikes, bumps, and cavities in a size range of about 1 nm to about 20 nm.

In some embodiments, the functionality of the active sites can originate, at least partially, from the structural features of the coating surface such as surface crystallinity, crystal grains size, and surface phase.

In some embodiments, the functionality of the active sites can originate, at least partially, from the combination of surface structure and composition.

In some embodiments, the localized active sites are located at the interface of coating and the pore.

In some embodiments, the active sites present in the microscopic pores include catalytic nanoparticles capable of chemically breaking down essential components of the viral protein structure and lead to viral inactivation or weakening.

In some embodiments, the catalytic nanoparticles include metal nanoparticles, such as gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, bimetals, metal alloys, metal compounds, such as pnictides, hydroxides, binary and complex salts, including heteropolyacids and their derivatives or a combination thereof.

In some embodiments, the catalytic nanoparticles include nanoparticles made of metal oxides, mixed metal oxides, and/or metal sulfide nanoparticles; some particular examples include vanadia, silica, alumina, titania, zirconia, hafnia, nickel oxide, cobalt oxide, tin oxide, manganese oxide, magnesium oxide, noble metal oxides, platinum group metal oxides, molybdenum oxides, tungsten oxides, rhenium oxides, tantalum oxide, niobium oxide, chromium oxides, scandium, yttrium, lanthanum, thorium, uranium oxides, other rare earth oxides, or a combination thereof.

In some embodiments, the catalytic nanoparticles include semiconductor nanoparticles, such as silicon or germanium, either pure or doped with elements or compounds of group III or V elements, or a combination thereof.

In some embodiments, an active site can include complex salts with alkali, alkali-earth, and group (III) metals and/or transition metal salts such as salts of nickel, copper, cobalt, manganese, magnesium, chromium, iron, platinum, tungsten, zinc, or other metals.

In some embodiments, an active site can include a metal cation, a metal oxide, organometallic complex or combination thereof.

In some embodiments, an active site can include a biologically derived material, such as an enzyme or a protein.

In some embodiments, the coating utilizes metal oxides that promote physisorption of the bioaerosols and particulates and their breakage.

In some embodiments, the activation of catalytic/functional sites can be achieved through heat or light activation.

In some embodiments, in use, the structure is maintained at elevated temperatures in the range of about 15° C. to about 500° C. In some embodiments, the structure is maintained at temperatures of about 20° C., about 30° C., about 40° C., about 50° C., about 60° C., about 70° C., about 80° C., about 90° C., about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., or about 475° C. Such elevated temperatures can facilitate the treatment (e.g., inactivation) of one or more contaminants due to activation of the active sites and/or thermal contact of the inner surface of the MS and the coating.

By way of example, the active sites can include metal nanoparticles composed of platinum group metals (e.g. Pd, Pt) which become catalytically active upon heating and induce oxidative damage to the surface of the pathogen and lead to its inactivation.

In some embodiments, the active components can be further designed to provide catalytic, photocatalytic, electrocatalytic, photonic, antimicrobial, light absorbing and/or emitting, stimuli responsiveness, adsorption, and desorption properties. The active sites can be introduced, for example, during the coating formation or through post modification.

In some embodiments, post modification comprises chemical modification of the surface of porous coating with active components including nanoparticles, chemical compounds, complexes through attachment of these functional units via covalent bonding, ionic bonding, van der Waals bonding and combination of thereof.

In some embodiments, the active material can be introduced through physical vapor deposition, atomic deposition, evaporation, spattering, wet chemical modification, ion impregnation, and any combination thereof.

In certain embodiments, the active sites are introduced via infiltration and/or adsorption into the coating.

### Macroscopic Structure (MS) Composition and Geometry

In some embodiments, the MS can include a material with macroscopic pores (e.g., “honeycomb” monolith structures, meshes, foams, sponges, textiles). In some embodiments, the MS can also include the nanostructured coatings covering at least 20%, or at least about 30%, or at least about 40%, or at least about 50%, or at least about 60%, or at least about 70%, or at least about 80%, or at least about 90%, or at least about 95%, or 100%, of the surface of the MS.

In certain embodiments, the MS can be made from a ceramic material, such as cordierite, Mullite, zeolite, and natural or synthetic clay.

In other embodiments, the MS can include a combination of composite metal and metal oxide, such as cermet.

In certain embodiments, the MS can be made from a metal salt or oxide, such as silica, alumina, alumina silicates, aluminum titanate, iron oxide, zinc oxide, tin oxide, beryllia, platinum group metal oxide, titania, zirconia, hafnia, molybdenum oxide, tungsten oxide, rhenium oxide, tantalum oxide, niobium oxide, vanadium oxide, chromium oxide, scandium oxide, yttria, lanthanum oxide, ceria, thorium oxide, uranium oxide, other rare earth oxides, and combinations thereof.

In certain embodiments, the MS can be made from one or more metals and/or metal alloys, such as stainless steel, ferritic steel (e.g., an iron-chromium alloy), austenitic steel (a chromium-nickel alloy), copper, nickel, brass, gold, silver, titanium, tungsten, aluminum, palladium, platinum, and combinations thereof.

In certain embodiments, the MS can include a semiconductor, including at least one of: silicon carbide, silicon, germanium, tin, silicon doped with a group III element, silicon doped with a group V element, germanium doped with a group III element, germanium doped with a group V element, tin doped with a group III element, tin doped with a group V element, and a transition metal oxide.

In certain embodiments, the MS can be made from a polymer, such as polyurethane, polystyrene, poly(methyl methacrylate), polyacrylate, poly(alkyl acrylate), substituted polyalkylacrylate, polystyrene, poly(divinylbenzene), polyvinylpyrrolidone, poly(vinyl alcohol), polyacrylamide, poly(ethylene oxide), polyvinyl chloride, polyvinylidene fluoride, polytetrafluoroethylene, other halogenated polymers, hydrogels, organogels, and combinations thereof. Other polymers of different architectures can be utilized as well, such as random and block copolymers, branched, star and dendritic polymers, and supramolecular polymers.

In certain embodiments, the MS can be made from one or more natural materials, such as cellulose, natural rubber (e.g. latex), wool, cotton, silk, linen, hemp, flax, and feather fiber.

In certain embodiments, the MS is composed of adjoint particulates. The particulates can be of arbitrary shape (e.g. spheroid, star-like, and elongated).

In certain embodiments, the MS can be made from natural or synthetic fabrics, textiles, paper, and any combinations thereof.

In some embodiments the MS can have a gyroid structure. In other embodiments the MS has a sponge-like structure.

In some embodiments, the porous macroscopic substrate can be in the form of a monolith, a metal mesh structure, a ceramic or a metal foam structure, a packed-bed structure, or HEPA type filter. In some embodiments, commercially available substrates or filters can be used as the porous macroscopic substrate, onto which the microscopic coatings are applied.

In some embodiments, the channels of the porous macroscopic substrate exhibit a geometry selected from the group consisting of a cylinder, a mesh, a foam, a spiral profile, a bead, woven or non-woven fibers-like structure, and woven fibers. The woven structure is weaved by interlacing warp and weft threads, and the non-woven structure is made by bonding fibers together by physical means, such as melt-blowing.

In some embodiments the structure of macroscopic substrate is designed to force air to flow through its porous internal walls (e.g. diesel particulate filter), for example, by plugging an end of the macroscopic channel. Accordingly, the chance for the air to contact the microscopic internal walls can be increased.

### Examples of Substrates Modified With Catalytic Coating

The following Examples are provided for further illustration of various aspects of the present teachings, and are not intended to indicate necessarily optimal ways of practicing the present teachings and/or optimal results that may be obtained.

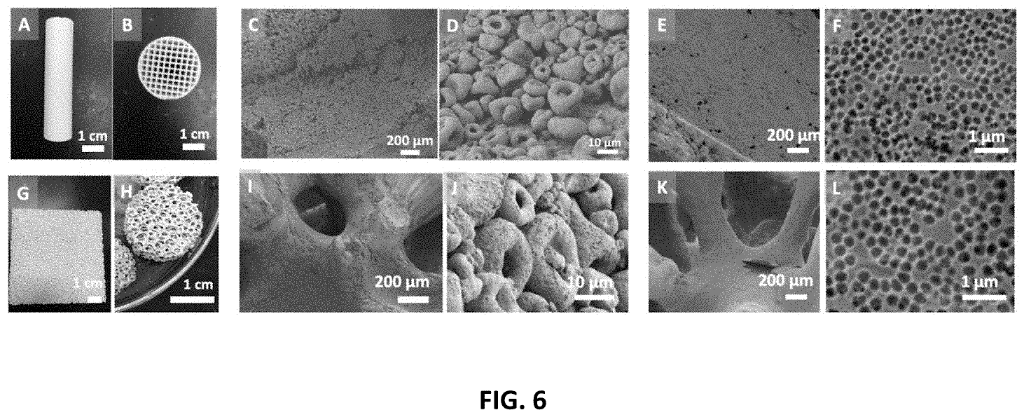

FIG. 6 shows representative images of two types of ceramic substrates, namely, open channels cordierite monolith (subpanels A and B) and ceramic foam (subpanels G and H). These substrates were modified with Pd/Al₂O₃ catalytic coating and the coating morphology was characterized with Scanning Electron Microscopy (SEM).

More specifically, subpanels A and B of FIG. 6 respectively show a side and a top views of an example of coated ceramic monolith including straight channels having 230 cells per square inch (cpsi). Subpanels C and D show the SEM images of internal channels of ceramic monolith at different magnifications coated with Pd/Al₂O₃ micropowders. Subpanels E and F show the SEM image of internal channels of ceramic monolith at different magnifications coated with Pd/Al₂O₃ nanoporous thin film.

Subpanels G and H of FIG. 6 respectively show a top view of ceramic foam from which a smaller circular sample was cut and modified with Pd/Al₂O₃ catalytic coating. Subpanels I and J show the SEM images of internal channels of ceramic foam at different magnifications coated with Pd/Al₂O₃ micropowders, and subpanels K and L show the SEM image of internal channels of ceramic foam at different magnifications coated with Pd/Al₂O₃ nanoporous thin film.

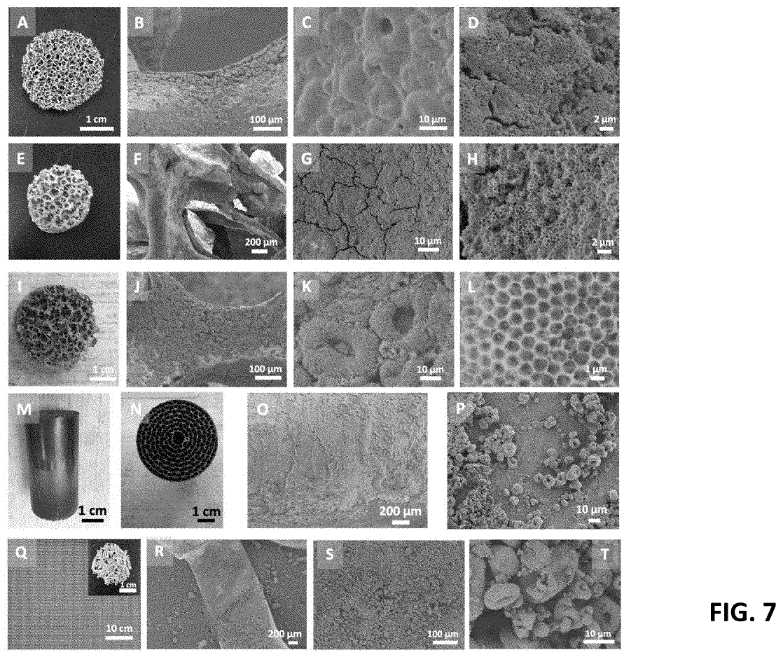

FIG. 7 shows representative images of five types of metallic substrates modified with Pd/Al₂O₃ catalytic coating. The coating morphology was characterized with Scanning Electron Microscopy (SEM). The five types of substrates include four types of metallic foams, namely, Ni, NiFe, FeCrAl, and a metallic (FeCrAl) corrugated monolith, as well as aluminum metallic mesh.

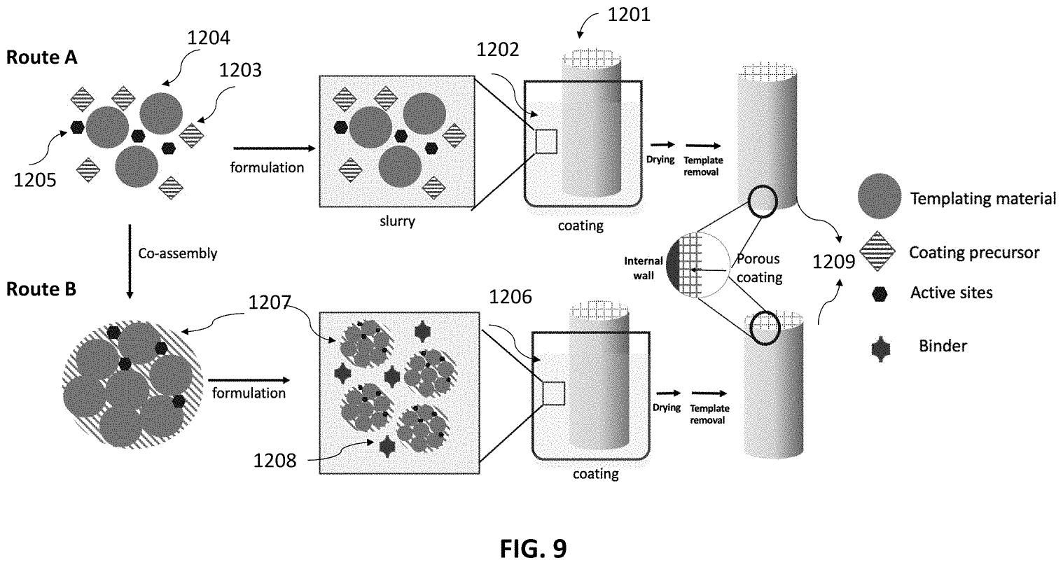

Subpanel A of FIG. 7 shows a representative Ni foam having 20 pores per inch (ppi). Subpanels B-D show the SEM images of internal channels of the foam at different magnifications coated with Pd/Al₂O₃ micropowders. Subpanel E of FIG. 9 shows a representative NiFe alloy foam (20 ppi). Subpanels F-H show the SEM images of internal channels of the NiFe foam at different magnifications coated with Pd/Al₂O₃ micropowders. Subpanel I of FIG. 7 shows a representative FeCrAl foam (40 ppi). Subpanels J-L show the SEM images of internal channels of the FeCrAl foam at different magnifications coated with Pd/Al₂O₃ micropowders. Subpanels M-N of FIG. 7 respectively show a side and a top views of a metallic corrugated monolith (FeCrAl, 400 cpsi). Subpanels O-P show the SEM images of internal channels of the FeCrAl metallic substrate at different magnifications coated with Pd/Al₂O₃ micropowders. Subpanel Q of FIG. 7 shows a representative aluminum mesh. Subpanels R-T show the SEM images of internal channels of the aluminum mesh at different magnifications coated with Pd/Al₂O₃ micropowders.

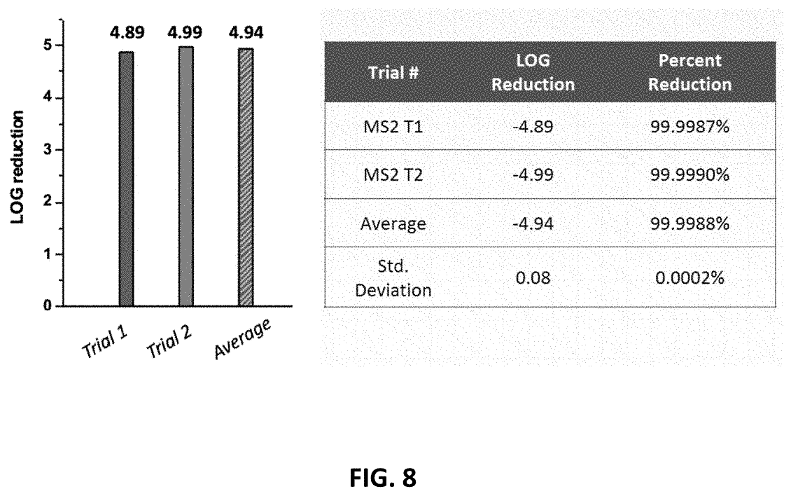

A prototype system according to the present teachings was fabricated to test the efficacy of a system according to an embodiment of the present teachings for inactivating aerosolized pathogens (e.g., live viruses or bio-aerosols). FIG. 8 depicts test results for virus deactivation using modified cordierite monolithic substrate in single pass experiment.

The prototype's effectiveness was tested against aerosolized MS2 Bacteriophage (MS2) in a single-pass configuration. In particular, MS2 is a viral RNA bacteriophage that is commonly used as a surrogate for the influenza virus and, according to the U.S. Food and Drug Administration (FDA), it also serves as a model surrogate for coronaviruses such as SARS-CoV-2. The efficacy of the device was assessed via an upstream and downstream sampling method to evaluate viable bioaerosol concentration in plaque-forming unit per volume (pfu/L). Comparison of the upstream and downstream samples yielded the single-pass efficiency in terms of the percent and LOG reduction of the bioaerosol challenge.

The results demonstrate that the prototype device achieves a significant reduction in viable MS2 bioaerosols. FIG. 8 presents the results of two trials for 140° C. of activation temperature, in particular, the amounts of reduction of the MS2 for the two trials, and an average and a standard deviation thereof. The results shown in FIG. 8 indicate that the device including a macroscopic structure (MS) according to the present teachings is capable of inactivating aerosolized live viruses with about 99.999% efficiency (i.e., about 5-log reduction or a 5 orders of magnitude reduction) in a single pass using the catalytic core operated at 140° C. and at a flow rate of 12.5 LPM.

### Fabrication of Material

One of the examples of a fabrication method according to an embodiment is described in FIG. 9. In this example, a macroscopic substrate (MS) 1201, with channels or pores as described above, is dipped into a composite coating components or slurry 1202 including coating precursors (or matrix precursor) 1203 (e.g., metal oxides), templating component 1204 and active component 1205.

In some embodiments, the slurry 1206 comprises preformed composite microparticles 1207 and a binder 1208. The microparticles 1207 are formed via co-assembly of templating component 1204, coating precursors 1203 and active component 1205 to form a composite coating.

After dipping, the MS 1201 is dried, then templating is removed through for example thermal treatment (calcination) or dissolution to give porous macroscopic substrate 1209, which can be formed from materials such as those described above. In some embodiments, the coating of the MS can be done through dip-coating, spray coating, and electrodeposition.

In some embodiments, the fabrication method can include spray coating of a macroscopic substrate 1201 with a composite coating components (slurry) to form a composite coating. In certain embodiments, the slurry can be applied via electrodeposition. In other embodiments, the slurry can be applied via brushing.

In some embodiments, the composite microparticles are formed through spray drying.

In some embodiments, the composite microparticles can have a size, e.g., a diameter, in the range of 5 micron to 80 microns.

In some embodiments, the coating precursor or slurry can include one of matrix precursor and templating material.

In some embodiments, the matrix precursor is a sol-gel, a nano-particulate, or a combination of thereof.

In certain embodiments, the sol-gel matrix precursor material is a silica, alumina, titania, and/or zirconia sol-gel and combination thereof.

In certain embodiments, the nano-particulate precursor comprises a single or a mixture of nanoparticles of the coating materials described above.

In some embodiments, the porous coating is formed through templating with sacrificial material.

In some embodiments, the process of removal of sacrificial templating material can include calcination, dissolution, etching, evaporation, sublimation, phase-separation, and combinations thereof.

In some embodiments, sacrificial templating material can be made from polymer colloids (e.g. polystyrene, PMMA). Many different types of colloidal particles can be utilized. The colloids can be made from various materials or mixtures of materials. In order to serve as sacrificial templating material, at least part of the colloidal material should be combustible, dissolvable, sublimable, or meltable during preparation of the coating.

In certain embodiments, the templating materials include polymeric fibers, biopolymer fibers, fibers with organometallic composition, supramolecular self-assembled fibers, or a combination thereof.

In certain embodiments, the templating particles include a colloidal dispersion of spherical, elongated, concave, amorphous, or facetted particles made from polymer, metal, metal oxides, supramolecular aggregates, crystals of organic, inorganic and organometallic compounds, or salts.

In certain embodiments, the templating materials can be made from a polymer, such as polyurethane, polystyrene, poly(methyl methacrylate), polyacrylate, poly(alkyl acrylate), substituted polyalkylacrylate, polystyrene, poly(divinylbenzene), polyvinylpyrrolidone, poly(vinylalcohol), polyacrylamide, poly(ethylene oxide), polyvinyl chloride, polyvinylidene fluoride, polytetrafluoroethylene, other halogenated polymers, hydrogels, organogels, and combinations thereof. Other polymers of different architectures can be utilized as well, such as random and block copolymers, branched, star and dendritic polymers, and supramolecular polymers.

In certain embodiments, the templating materials can be made from one or more natural materials, such as cellulose, natural rubber (e.g. latex), wool, cotton, silk, linen, hemp, flax, and feather fiber.

In certain embodiments, the templating materials can be made from natural or synthetic fabrics and textiles, and combinations thereof.

In some embodiments, active components can be introduced after fabrication of structured material through the post modification of the surface of the porous coating. For example, by a chemical modification of the coating surface following the attachment of catalytic nanoparticles.

What is claimed is:

1. A material structure, comprising:
a macroscopic porous substrate configured to receive a flow of a medium for passage of at least a portion thereof through the porous substrate,
at least one porous coating disposed on at least a portion of an inner surface of said porous substrate,
wherein said porous coating comprises a matrix having a plurality of interconnected passages, and
wherein said macroscopic porous substrate and said coating are configured to treat at least one contaminant, if any, present said flowing medium.

2. The material structure of claim 1, wherein at least a portion of an inner surface of at least one of said passages of the coating comprises an active site suitable for treating said contaminant.

3. The material structure of claim 2, wherein said active site comprises catalytically active material.

4. The material structure of claim 3, wherein said catalytically active material is any of a thermally, photocatalytically and electro-catalytically active material.

5. The material structure of claim 3, wherein said catalytically active material comprises any of oxides, mixed oxides, mixed oxides of elements from one or more groups I, II, III, IV V, VI, zeolites, oxohydroxides, aluminates, silicates, alumosilicates, titanates, oxometallates, metal-organic frameworks, vanadia, silica, alumina, titania, zirconia, hafnia, nickel oxide, cobalt oxide, copper oxide, tin oxide, manganese oxide, magnesium oxide, silver oxide, noble metal oxides, platinum group metal oxides, molybdenum oxides, tungsten oxides, rhenium oxides, tantalum oxide, niobium oxide, chromium oxides, scandium, yttrium, lanthanum, thorium, rare earth oxides, or a combination thereof.

6. The material structure of claim 1, wherein said macroscopic porous substrate and said porous coating are configured to provide an entrapment of said at least one contaminant.

7. The material structure of claim 1, wherein said macroscopic porous substrate and said porous coating are configured to treat at least a portion of one or more particulates, if any, present in said flowing medium.

8. The material structure of claim 1, wherein said macroscopic porous substrate and said porous coating are configured to treat at least a portion of one or more bioaerosols, if any, present in said flowing medium.

9. The material structure of claim 1, wherein said macroscopic porous substrate and said porous coating are configured to treat at least a portion of one or more types of pathogenic organisms, if any, present in said flowing medium.

10. The material structure of claim 9, wherein said at least one pathogenic microorganism comprises any of one or more viruses, bacteria, and fungi present in said flowing medium.

11. The material structure of claim 1, wherein said medium comprises contaminated air.

12. The material structure of claim 1, wherein said contaminant comprises at least one airborne contaminant.

13. The material structure of claim 1, wherein said medium comprises a liquid.

14. The material structure of claim 13, wherein said liquid comprises any of a water-based liquid, an aqueous dispersion, an organic liquid, an organic dispersion, and an ionic liquid.

15. The material structure of claim 1, wherein said macroscopic porous substrate and said porous coating are configured to provide at least temporary entrapment of said at least one contaminant.

16. The material structure of claim 15, wherein said passages of the coating exhibit a geometry, a surface roughness and a size configured to facilitate said entrapment of said at least one contaminant.

17. The material structure of claim 1, wherein said coating exhibits any of inverse opal structure, sponge-like, and gyroid geometry.

18. The material structure of claim 1, wherein said coating exhibits a thickness in a range of about 1 to about 200 micrometers.

19. The material structure of claim 1, wherein said interconnected passages of the coating exhibit a cross-sectional dimension in a range of about 100 nm to about 20 microns.

20. The material structure of claim 1, wherein said interconnected passages of the coating exhibit a cross-sectional dimension in a range of about 200 nm to about 10 microns.

21. The material structure of claim 1, wherein said interconnected passages of the coating exhibit a cross-sectional dimension in a range of about 300 nm to about 5 microns.

22. The material structure of claim 1, wherein said interconnected passages of the coating exhibit a surface area in the range of about 10 m²/g to about 500 m²/g.

23. The material structure of claim 1, wherein said interconnected passages of the coating exhibit a cross-sectional size that is equal to or greater than an average size of said at least one contaminant and less than about a hundred times of the average size of said at least one contaminant.

24. The material structure of claim 1, wherein said coating comprises any of oxides, mixed oxides, mixed oxides of elements from one or more groups I, II, III, IV V, VI, zeolites, oxohydroxides, aluminates, silicates, alumosilicates, titanates, oxometallates, metal-organic frameworks, vanadia, silica, alumina, titania, zirconia, hafnia, nickel oxide, cobalt oxide, cobalt oxide, tin oxide, manganese oxide, magnesium oxide, noble metal oxides, platinum group metal oxides, molybdenum oxides, tungsten oxides, rhenium oxides, tantalum oxide, niobium oxide, chromium oxides, scandium, yttrium, lanthanum, thorium, rare earth oxides or a combination thereof.

25. The material structure of claim 1, wherein said coating comprises any of a synthetic polymer, a natural polymer, a bio-polymer or a combination thereof.

26. The material structure of claim 1, wherein said macroscopic substrate is configured to cause turbulence in said flow of the medium therethrough.

27. The material structure of claim 1, wherein said macroscopic porous substrate and said coating are configured such that said flowing medium therethrough results in treatment of at least about 70% of said at least one contaminant.

28. The material structure of claim 1, wherein said macroscopic porous substrate and said coating are configured such that said flowing medium therethrough results in treatment of at least about 80% of said at least one contaminant.

29. The material structure of claim 1, wherein said macroscopic porous substrate and said coating are configured such that said flowing medium therethrough results in treatment of at least about 90% of said at least one contaminant.

30. The material structure of claim 1, wherein said macroscopic porous substrate and said coating are configured such that said flowing medium therethrough results in treatment of at least about 95% of said at least one contaminant.

31. The material structure of claim 1, wherein said macroscopic porous substrate and said coating are configured such that said flowing medium therethrough results in treatment of at least about 99% of said at least one contaminant.

32. The material structure of claim 1, wherein said coating comprises a continuous film.

33. The material structure of claim 1, wherein said coating comprises a plurality of discontinuous segments.

34. The material structure of claim 1, wherein said macroscopic porous substrate comprises one or more channels that have an average cross-sectional dimension in a range of about 50 microns to about 10,000 microns.

35. The material structure of claim 34, wherein said one or more channels exhibit a length in a range of about 1 mm to about 1 m.

36. The material structure of claim 1, wherein said macroscopic porous substrate comprises any of a ceramic, a metal, a metallic alloy, a carbide, a metal felt, FeCrAl, natural clay, a polymeric material and combinations thereof.

37. The material structure of claim 36, wherein said ceramic comprises a cordierite.

38. The material structure of claim 1, wherein said macroscopic porous substrate comprises a particulate filter.

39. The material structure of claim 34, wherein said one or more channels of said macroscopic porous substrate exhibit a geometry selected from the group consisting of a cylinder, a mesh, a foam, a spiral profile, a bead, and woven or non-woven fibers-like structure.

40. The material structure of claim 34, wherein said one or more channels of the macroscopic porous substrate are arranged relative to one another as any of a plurality of parallel channels, randomly oriented channels, interconnected or isolated channels, a sponge-like configuration, a corrugated geometry, a spiral geometry and any combination thereof.

41. The material structure of claim 2, wherein said active sites comprise any of a metal, one or more metal alloys, a multimetallic species, a metal cation, a metal sulfide, a binary metal salt, a metal salt of transition metals, a complex metal salt, a metal salt of an organic acid, a metal salt of inorganic acid, a metal salt of a complex acid, a base, an acid, organometallic complexes, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, metal oxides, mixed metal oxides, iron oxides, cobalt oxides, nickel oxides, manganese oxide, magnesium oxide, noble metal oxides, ruthenium oxides, rhodium oxides, palladium oxides, osmium oxides, iridium oxides, platinum oxides, copper oxides, silver oxides gold oxides, vanadium oxides, niobium oxide, tantalum oxide, chromium oxide, molybdenum oxide, tungsten oxide, scandium oxide, yttrium oxide, lanthanum oxide, rare earth metal oxide, and any combinations thereof.

42. The material structure of claim 2, wherein said active sites comprise a plurality of nanoparticles.

43. The material structure of claim 42, wherein said nanoparticles comprise any of metal, multiple metals, a metal alloy, gold, silver, platinum, palladium, ruthenium, rhodium, cobalt, iron, nickel, osmium, iridium, rhenium, copper, chromium, tungsten, molybdenum, vanadium, niobium, tantalum, titanium, zirconium, hafnium, bimetals, metal alloys, a metal oxide, a mixed metal oxide, a metal sulfide, a binary metal salt, a complex metal salt, a metal salt of an organic acid, a metal salt of inorganic acid, a metal salt of a complex acid, a base, an acid, an organometallic compound, a coordination compound, one or more platinum group metal oxides, silica, alumina, iron oxides, cobalt oxides, nickel oxides, ruthenium oxides, rhodium oxides, palladium oxides, osmium oxides, iridium oxides, platinum oxides, copper oxides, silver oxides, gold oxides, vanadium oxides, zirconium oxide, cerium oxide, manganese oxide, magnesium oxide, niobium oxide, tantalum oxide, chromium oxide, molybdenum oxide, tungsten oxide, scandium oxide, yttrium oxide, lanthanum oxide, rare earth metal oxide, and any combinations thereof.

44. The material structure of claim 2, wherein said active sites comprise a biological agent.

45. The material structure of claim 44, wherein said biological agent comprises a protein that is chemically or physically coupled to an internal surface portion of said coating.

46. The material structure of claim 45, wherein said protein comprises an enzyme.

47. The material structure of claim 2, wherein said active sites comprise semiconductor nanoparticles doped with any of group III and group V elements, or a combination thereof.

48. The material structure of claim 2, wherein said active sites are configured to be activated via any of heat and radiation.

49. The material structure of claim 48, wherein said active sites are configured to be activated by raising a temperature thereof to a range of about 15° C. to about 500° C.

50. The material structure of claim 48, wherein said radiation has a wavelength in a range of microwave, ultraviolet (UV), visible, and infrared (IR) portions of the electromagnetic spectrum and combinations thereof.

51. The material structure of claim 50, wherein said radiation has a wavelength in a range of about 160 nm to about 1500 nm.

52. The material structure of claim 2, wherein said active sites are configured to provide at least one of a catalytic, a photonic, an antimicrobial, a light-absorbing, a light-emitting, a stimuli responsiveness, an adsorption, a desorption property and combination thereof.

53. The material structure of claim 52, wherein said catalytic property comprises any of a photocatalytic and an electrocatalytic property.

54. An air purifier comprising the material structure of claim 1.

55. A water purifier comprising the material structure of claim 1.
